(19) United States
(12) Patent Application Publication
(10) Pub. No.: US 2015/0290032 A1
(43) Pub. Date: Oct. 15, 2015

(54) MANIFEST REFRACTION TREATMENT SYSTEMS AND METHODS

(71) Applicant: AMO Development, LLC, Santa Ana, CA (US)

(72) Inventor: Michael Brownell, San Clemente, CA (US)

(21) Appl. No.: 14/701,037

(22) Filed: Apr. 30, 2015

Related U.S. Application Data
(63) Continuation of application No. 14/233,730.
(60) Provisional application No. 61/509,669, filed on Jul. 20, 2011.

(51) Int. Cl. A61F 9/008; A61B 3/10; A61B 3/04; A61B 3/107; A61B 3/028; A61B 3/103; A61B 3/00

(52) U.S. Cl. CPC A61F 9/008; A61B 3/103; A61B 3/1015; A61B 3/0025; A61B 3/107; A61B 3/028; A61B 3/0285; A61B 3/04; A61F 2009/0088; A61F 2009/00882

(57) ABSTRACT

Embodiments of the present invention encompass systems and techniques for developing a vision prescription for a patient based on an objective optical manifest refraction, such as that measured with a wavefront device, without altering the prescription in response to any subjective manifest refraction, such as that measured with a phoropter device.

### CROSS-REFERENCES TO RELATED APPLICATIONS

This application is a continuation of U.S. patent application Ser. No. 14/233,730, filed Jan. 17, 2014, which is a 371 national stage patent application of PCT International Patent Application No. PCT/US2012/047655, filed on Jul. 20, 2012, which is a nonprovisional of and claims the benefit of priority to U.S. Provisional Patent Application No. 61/509,669, filed Jul. 20, 2011. This application is also related to U.S. patent application Ser. Nos. 12/418,841 filed Apr. 6, 2009, and 61/428,644 filed Dec. 30, 2010. The contents of each of the aforementioned applications are incorporated herein by reference in their entirety.

### BACKGROUND OF THE INVENTION

Embodiments of the present invention relate to systems and methods for vision correction, and in particular to techniques for planning a prescription treatment for a patient's eye.

As light rays enter the eye, they are bent by the anterior portion of the eye before reaching the retina. This refraction of the light is a consequence of the optical power of the cornea and lens. If there are refractive errors in the eye, incoming light does not properly converge at the retina. For example, the eye may present spherical or cylindrical irregularities that prevent the proper focusing of light. Manifest refraction, such as that measured by a phoropter device, is an indication of how much spherical and cylindrical error shows up as a person perceives vision. Currently available vision treatment approaches often rely upon manually measured subjective manifest refraction for determining or adjusting a patient prescription. For example, a treatment provider can manually evaluate the subjective manifest refraction of the patient corresponding to a spectacle correction, convert that manifest refraction to the corneal plane using a vertex distance adjustment, and use the resulting manifest refraction for determining the prescription.

Although these and other proposed vision treatment devices and methods may provide real benefits to patients in need thereof, still further advances would be desirable. For example, there continues to be a need for improved treatment systems and methods that provide for the planning of vision prescriptions that deliver enhanced optical performance. Embodiments of the present invention provide solutions that address certain inefficiencies or shortcomings which may be associated with known techniques, and hence provide answers to at least some of these outstanding needs.

### BRIEF SUMMARY OF THE INVENTION

Embodiments of the present invention encompass systems and methods for determining a vision prescription or planning a refractive treatment for a patient, based on a pre-treatment refractive measurement, without altering the prescription in response to any measured subjective manifest refraction. In some instances, subjective manifest refraction may be measured but not factored into the prescription generation process. That can be the case even where the subjective manifest refraction measurement differs significantly from the objective manifest refraction measurement. In some instances, subjection manifest refraction may be measured and used as a safety check. Optionally, a prescription can be generated for a patient based on an objective optical manifest refraction measurement, without taking a measurement of the subjective manifest refraction at all. Such techniques are well suited for use in any of a variety of vision treatment modalities based on manifest refraction measurements, including without limitation laser ablation treatments, contact lens treatments, spectacle treatments, surgical vision treatments or modifications, intraocular lens and custom intraocular lens treatments, and the like.

In one aspect, embodiments of the present invention encompass methods and systems for treating an eye of a patient. An exemplary method includes measuring, with a manifest refraction instrument, an objective optical manifest refraction of the eye of the patient, transmitting the objective optical manifest refraction measurement from the manifest refraction instrument to a treatment planner, determining, with the treatment planner, a prescription based on the objective optical manifest refraction measurement, without altering the prescription in response to any subjective manifest refraction measurement of the eye, and transmitting the prescription from the treatment planner so as to facilitate surgically altering the eye per the prescription. In some cases, the objective optical manifest refraction measurement includes a wavefront evaluation of the eye. In some cases, the objective optical manifest refraction measurement includes a combined wavefront and topographic evaluation of the eye. In some cases, methods may include interactively, in response to subjective input from the patient, measuring the subjective manifest refraction of the eye, the subjective manifest refraction measurement differing significantly from the optical manifest refraction measurement. In some cases, the objective optical manifest refraction measurement transmitted from the manifest refraction instrument may differ from the subjective manifest refraction measurement by more than about 0.10 Diopters. In some cases, the objective optical manifest refraction measurement transmitted from the manifest refraction instrument may differ from the subjective manifest refraction measurement by more than about 0.25 Diopters. In some cases, the objective optical manifest refraction measurement transmitted from the manifest refraction instrument may differ from the subjective manifest refraction measurement by more than about 0.37 Diopters. In some cases, the objective optical manifest refraction measurement transmitted from the manifest refraction instrument may differ from the subjective manifest refraction measurement by more than about 0.50 Diopters. Optionally, the subjective manifest refraction measurement includes a phoropter evaluation of the eye. In some instances, the subjective manifest refraction measurement includes a trial frame evaluation of the eye. In some instances, the subjective manifest refraction measurement includes a trial lens evaluation of the eye. According to some embodiments, the objective optical manifest refraction includes an objective optical sphere value, and the subjective manifest refraction includes a subjective sphere value that differs from the optical sphere value by greater than about 0.3 Diopters. In some cases, the difference may be between about 0.5 Diopters and about 1.0 Diopters. In some cases, the difference may be greater than about 0.5 Diopters. Methods may further include repeating at least one of the subjective manifest refraction measurement and the objective manifest refraction measurement in response to the difference in objective optical and subjective sphere values. Methods may also include determining and imposing the prescription on the eye without altering the objective optical manifest refraction or the transmitted prescription in response to the subjective manifest refraction measurement(s). According to some embodiments, the eye is surgically altered under direction of a treating physician Relatedly, the eye can be surgically altered without the treating physician measuring subjective manifest refraction of the eye or otherwise obtaining the subjective manifest refraction of the eye in preparation for the surgical alteration.

In another aspect, embodiments of the present invention encompass methods for treating an eye of a patient, which include measuring, with a manifest refraction instrument, an objective optical manifest refraction of the eye of the patient, transmitting the objective optical manifest refraction measurement from the manifest refraction instrument to a treatment planner, reviewing a subjective manifest refraction measurement of the eye, where the subjective manifest refraction differing significantly from the objective manifest refraction, determining, with the treatment planner, a prescription based on the objective optical manifest refraction measurement, without altering the prescription in response to the subjective manifest refraction measurement of the eye, and transmitting the prescription from the treatment planner so as to facilitate surgically altering the eye per the prescription. In some instances, he patient remains well rested throughout the duration in which the subjective manifest refraction measurement of the eye is performed. The subjective manifest refraction measurement can be determined using a phoropter having a lens, where the patient is able to effectively evaluate vision provided by the lens, where the patient is in good health and able to provide sufficient input to effectively determine the subjective manifest refraction measurement, and where the patient is mature enough to be able to provide sufficient input to effectively determine the subjective manifest refraction measurement.

In still another aspect, embodiments of the present invention encompass methods for treating an eye of a patient which include receiving an objective optical manifest refraction of the eye of the patient, transmitting the objective optical manifest refraction measurement from the manifest refraction instrument to a treatment planner, determining, with the treatment planner, a prescription based on the objective optical manifest refraction measurement, without altering the prescription in response to any subjective manifest refraction measurement of the eye, and transmitting the prescription from the treatment planner so as to facilitate surgically altering the eye per the prescription. In some cases, methods may further include measuring the objective optical manifest refraction of the eye of the patient.

In yet another aspect, embodiments of the present invention encompass systems for deriving a prescription for an eye of a patient. Exemplary systems may include a manifest refraction instrument that measures an objective optical manifest refraction of the eye of the patient, and a treatment planner that determines a prescription based on the objective optical manifest refraction measurement, without altering the prescription in response to any subjective manifest refraction measurement of the eye. The treatment planner can be coupled with the manifest refraction instrument so as to receive the objective optical manifest refraction therefrom. Systems may also include a surgical device that alters the eye per the prescription. The surgical device can be coupleable with the treatment planner so as to receive the prescription from the treatment planner. In some instances, the objective optical manifest refraction measurement includes a wavefront evaluation of the eye. In some instances, the objective optical manifest refraction measurement includes a combined wavefront and topographic evaluation of the eye. In some instances, the treatment planner can be configured to determine the prescription based on the objective optical manifest refraction measurement when the subjective manifest refraction measurement differs significantly from the optical manifest refraction measurement. In some instances, the treatment planner can be configured to determine the prescription based on the objective optical manifest refraction measurement when the objective optical manifest refraction measurement differs from the subjective manifest refraction measurement by more than about 0.10 Diopters. In some instances, the treatment planner can be configured to determine the prescription based on the objective optical manifest refraction measurement when the objective optical manifest refraction measurement differs from the subjective manifest refraction measurement by more than about 0.25 Diopters. In some instances, the treatment planner can be configured to determine the prescription based on the objective optical manifest refraction measurement when the objective optical manifest refraction measurement differs from the subjective manifest refraction measurement by more than about 0.37 Diopters. In some instances, the treatment planner can be configured to determine the prescription based on the objective optical manifest refraction measurement when the objective optical manifest refraction measurement differs from the subjective manifest refraction measurement by more than about 0.50 Diopters. In some instances, the treatment planner can be configured to determine the prescription based on the objective optical manifest refraction measurement when the objective optical manifest refraction measurement differs from the subjective manifest refraction measurement by more than about one standard deviation associated with the subjective manifest refraction measurement.

In another aspect, embodiments of the present invention encompass systems for deriving a prescription for an eye of a patient that include a wavefront-based instrument that measures a wavefront-derived objective optical manifest refraction measurement of the eye of the patient. The wavefront-derived manifest refraction can include a sphere component, a cylinder component, and an axis component. Systems may also include a treatment planner that determines a prescription based on the objective optical manifest refraction measurement, without altering the prescription in response to any subjective manifest refraction measurement of the eye. Systems may also include a surgical device that alters the eye per the prescription. In some cases, systems can include a memory that receives the subjective manifest refraction measurement of the eye. In some cases, the wavefront-based instrument can include the memory. In some cases, the treatment planner can include the memory. In some cases, the surgical device can include the memory. According to some embodiments, systems may include a processor that calculates a difference between the wavefront-derived objective optical manifest refraction measurement and the subjective manifest refraction measurement. Systems may also include a prompting mechanism that presents a signal if the difference exceeds a threshold. According to some embodiments, systems may include an input that receives a physician acknowledgement of the difference. According to some embodiments, systems may include an input that receives a re-measurement instruction from the physician.

For a fuller understanding of the nature and advantages of the present invention, reference should be had to the ensuing detailed description taken in conjunction with the accompanying drawings.

### BRIEF DESCRIPTION OF THE DRAWINGS

FIG. 1 illustrates a laser ablation system according to an embodiment of the present invention.

FIG. 2 illustrates a simplified computer system according to an embodiment of the present invention.

FIG. 3 illustrates a wavefront measurement system according to an embodiment of the present invention.

FIG. 3A illustrates another wavefront measurement system according to an embodiment of the present invention.

FIG. 4 illustrates aspects of prescription methods according to embodiments of the present invention.

FIGS. 5A and 5B depict aspects of refraction study results according to embodiments of the present invention.

FIG. 6 depicts aspects of refraction study results according to embodiments of the present invention.

FIG. 7 depicts aspects of refraction study results according to embodiments of the present invention.

FIG. 8 depicts aspects of refraction study results according to embodiments of the present invention.

FIG. 9 depicts aspects of refraction study results according to embodiments of the present invention.

FIG. 10 depicts aspects of refraction study results according to embodiments of the present invention.

FIG. 11 depicts aspects of refraction study results according to embodiments of the present invention.

FIGS. 12A and 12B depict aspects of refraction study results according to embodiments of the present invention.

### DETAILED DESCRIPTION OF THE INVENTION

Exemplary systems and methods as described herein involve the use of an objective optical manifest refraction, such as that measured with a wavefront device, for developing a vision prescription for a patient without altering the prescription in response to any subjective manifest refraction, such as that measured with a phoropter device.

Embodiments of the present invention can be readily adapted for use with existing laser systems and other optical treatment devices. Although system, software, and method embodiments of the present invention are described primarily in the context of a laser eye surgery system, it should be understood that embodiments of the present invention may be adapted for use in alternative eye treatment procedures, systems, or modalities, such as spectacle lenses, intraocular lenses, accommodating IOLs, contact lenses, corneal ring implants, collagenous corneal tissue thermal remodeling, corneal inlays, corneal onlays, other corneal implants or grafts, and the like. Relatedly, systems, software, and methods according to embodiments of the present invention are well suited for customizing any of these treatment modalities to a specific patient. Thus, for example, embodiments encompass custom intraocular lenses, custom contact lenses, custom corneal implants, and the like, which can be configured to treat or ameliorate any of a variety of vision conditions in a particular patient based on their unique ocular characteristics or anatomy.

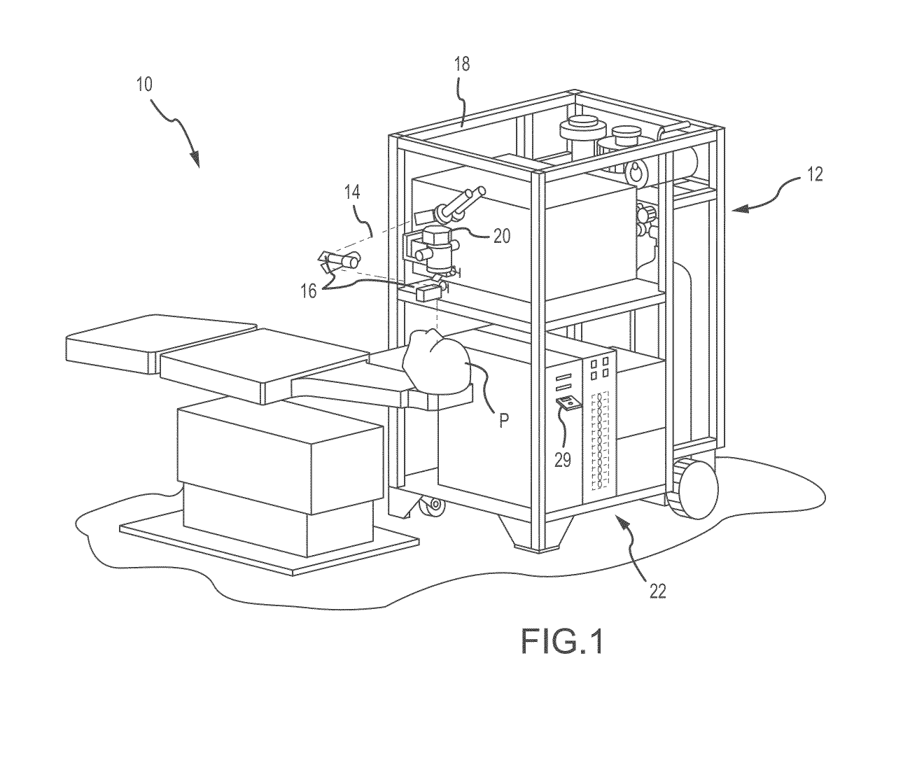

Turning now to the drawings, FIG. 1 illustrates a laser eye surgery system 10 of the present invention, including a laser 12 that produces a laser beam 14. Laser 12 is optically coupled to laser delivery optics 16, which directs laser beam 14 to an eye E of patient P. A delivery optics support structure (not shown here for clarity) extends from a frame 18 supporting laser 12. A microscope 20 is mounted on the delivery optics support structure, the microscope often being used to image a cornea of eye E.

Laser 12 generally comprises an excimer laser, ideally comprising an argon-fluorine laser producing pulses of laser light having a wavelength of approximately 193 nm. Laser 12 will preferably be designed to provide a feedback stabilized fluence at the patient's eye, delivered via delivery optics 16. The present invention may also be useful with alternative sources of ultraviolet or infrared radiation, particularly those adapted to controllably ablate the corneal tissue without causing significant damage to adjacent and/or underlying tissues of the eye. Such sources include, but are not limited to, solid state lasers and other devices which can generate energy in the ultraviolet wavelength between about 185 and 205 nm and/or those which utilize frequency-multiplying techniques. Hence, although an excimer laser is the illustrative source of an ablating beam, other lasers may be used in the present invention.

Laser system 10 will generally include a computer or programmable processor 22. Processor 22 may comprise (or interface with) a conventional PC system including the standard user interface devices such as a keyboard, a display monitor, and the like. Processor 22 will typically include an input device such as a magnetic or optical disk drive, an internet connection, or the like. Such input devices will often be used to download a computer executable code from a tangible storage media 29 embodying any of the methods of the present invention. Tangible storage media 29 may take the form of a floppy disk, an optical disk, a data tape, a volatile or non-volatile memory, RAM, or the like, and the processor 22 will include the memory boards and other standard components of modern computer systems for storing and executing this code. Tangible storage media 29 may optionally embody wavefront sensor data, wavefront gradients, a wavefront elevation map, a treatment map, a corneal elevation map, and/or an ablation table. While tangible storage media 29 will often be used directly in cooperation with a input device of processor 22, the storage media may also be remotely operatively coupled with processor by means of network connections such as the internet, and by wireless methods such as infrared, Bluetooth, or the like.

Laser 12 and delivery optics 16 will generally direct laser beam 14 to the eye of patient P under the direction of a computer 22. Computer 22 will often selectively adjust laser beam 14 to expose portions of the cornea to the pulses of laser energy so as to effect a predetermined sculpting of the cornea and alter the refractive characteristics of the eye. In many embodiments, both laser beam 14 and the laser delivery optical system 16 will be under computer control of processor 22 to effect the desired laser sculpting process, with the processor effecting (and optionally modifying) the pattern of laser pulses. The pattern of pulses may by summarized in machine readable data of tangible storage media 29 in the form of a treatment table, and the treatment table may be adjusted according to feedback input into processor 22 from an automated image analysis system in response to feedback data provided from an ablation monitoring system feedback system. Optionally, the feedback may be manually entered into the processor by a system operator. Such feedback might be provided by integrating the wavefront measurement system described below with the laser treatment system 10, and processor 22 may continue and/or terminate a sculpting treatment in response to the feedback, and may optionally also modify the planned sculpting based at least in part on the feedback. Measurement systems are further described in U.S. Pat. No. 6,315,413, the full disclosure of which is incorporated herein by reference.

Laser beam 14 may be adjusted to produce the desired sculpting using a variety of alternative mechanisms. The laser beam 14 may be selectively limited using one or more variable apertures. An exemplary variable aperture system having a variable iris and a variable width slit is described in U.S. Pat. No. 5,713,892, the full disclosure of which is incorporated herein by reference. The laser beam may also be tailored by varying the size and offset of the laser spot from an axis of the eye, as described in U.S. Pat. Nos. 5,683,379, 6,203,539, and 6,331,177, the full disclosures of which are incorporated herein by reference.

Still further alternatives are possible, including scanning of the laser beam over the surface of the eye and controlling the number of pulses and/or dwell time at each location, as described, for example, by U.S. Pat. No. 4,665,913, the full disclosure of which is incorporated herein by reference; using masks in the optical path of laser beam 14 which ablate to vary the profile of the beam incident on the cornea, as described in U.S. Pat. No. 5,807,379, the full disclosure of which is incorporated herein by reference; hybrid profile-scanning systems in which a variable size beam (typically controlled by a variable width slit and/or variable diameter iris diaphragm) is scanned across the cornea; or the like. The computer programs and control methodology for these laser pattern tailoring techniques are well described in the patent literature.

Additional components and subsystems may be included with laser system 10, as should be understood by those of skill in the art. For example, spatial and/or temporal integrators may be included to control the distribution of energy within the laser beam, as described in U.S. Pat. No. 5,646,791, the full disclosure of which is incorporated herein by reference. Ablation effluent evacuators/filters, aspirators, and other ancillary components of the laser surgery system are known in the art. Further details of suitable systems for performing a laser ablation procedure can be found in commonly assigned U.S. Pat. Nos. 4,665,913, 4,669,466, 4,732,148, 4,770,172, 4,773,414, 5,207,668, 5,108,388, 5,219,343, 5,646,791 and 5,163,934, the complete disclosures of which are incorporated herein by reference. Suitable systems also include commercially available refractive laser systems such as those manufactured and/or sold by Alcon, Bausch & Lomb, Nidek, WaveLight, LaserSight, Schwind, Zeiss-Meditec, and the like. Basis data can be further characterized for particular lasers or operating conditions, by taking into account localized environmental variables such as temperature, humidity, airflow, and aspiration.

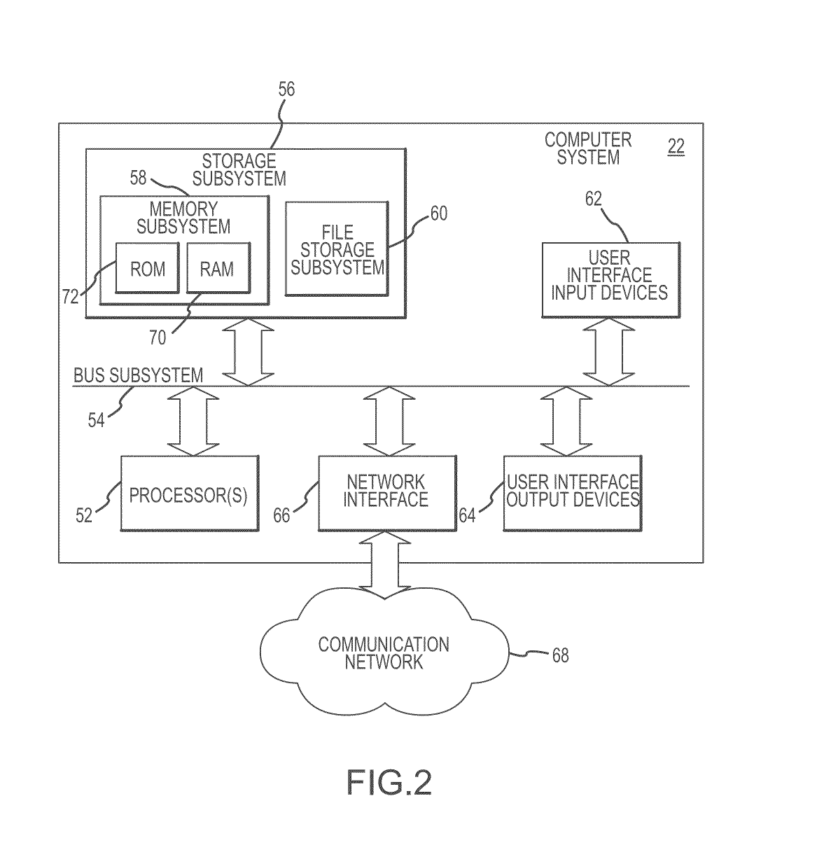

FIG. 2 is a simplified block diagram of an exemplary computer system 22 that may be used by the laser surgical system 10 of the present invention. Computer system 22 typically includes at least one processor 52 which may communicate with a number of peripheral devices via a bus subsystem 54. These peripheral devices may include a storage subsystem 56, comprising a memory subsystem 58 and a file storage subsystem 60, user interface input devices 62, user interface output devices 64, and a network interface subsystem 66. Network interface subsystem 66 provides an interface to outside networks 68 and/or other devices, such as the wavefront measurement system 30.

User interface input devices 62 may include a keyboard, pointing devices such as a mouse, trackball, touch pad, or graphics tablet, a scanner, foot pedals, a joystick, a touchscreen incorporated into the display, audio input devices such as voice recognition systems, microphones, and other types of input devices. User input devices 62 will often be used to download a computer executable code from a tangible storage media 29 embodying any of the methods of the present invention. In general, use of the term “input device” is intended to include a variety of conventional and proprietary devices and ways to input information into computer system 22.

User interface output devices 64 may include a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from computer system 22 to a user.

Storage subsystem 56 can store the basic programming and data constructs that provide the functionality of the various embodiments of the present invention. For example, a database and modules implementing the functionality of the methods of the present invention, as described herein, may be stored in storage subsystem 56. These software modules are generally executed by processor 52. In a distributed environment, the software modules may be stored on a plurality of computer systems and executed by processors of the plurality of computer systems. Storage subsystem 56 typically comprises memory subsystem 58 and file storage subsystem 60.

Memory subsystem 58 typically includes a number of memories including a main random access memory (RAM) 70 for storage of instructions and data during program execution and a read only memory (ROM) 72 in which fixed instructions are stored. File storage subsystem 60 provides persistent (non-volatile) storage for program and data files, and may include tangible storage media 29 (FIG. 1) which may optionally embody wavefront sensor data, wavefront gradients, a wavefront elevation map, a treatment map, and/or an ablation table. File storage subsystem 60 may include a hard disk drive, a floppy disk drive along with associated removable media, a Compact Digital Read Only Memory (CD-ROM) drive, an optical drive, DVD, CD-R, CD-RW, solid-state removable memory, and/or other removable media cartridges or disks. One or more of the drives may be located at remote locations on other connected computers at other sites coupled to computer system 22. The modules implementing the functionality of the present invention may be stored by file storage subsystem 60.

Bus subsystem 54 provides a mechanism for letting the various components and subsystems of computer system 22 communicate with each other as intended. The various subsystems and components of computer system 22 need not be at the same physical location but may be distributed at various locations within a distributed network. Although bus subsystem 54 is shown schematically as a single bus, alternate embodiments of the bus subsystem may utilize multiple busses.

Computer system 22 itself can be of varying types including a personal computer, a portable computer, a workstation, a computer terminal, a network computer, a control system in a wavefront measurement system or laser surgical system, a mainframe, or any other data processing system. Due to the ever-changing nature of computers and networks, the description of computer system 22 depicted in FIG. 2 is intended only as a specific example for purposes of illustrating one embodiment of the present invention. Many other configurations of computer system 22 are possible having more or less components than the computer system depicted in FIG. 2.

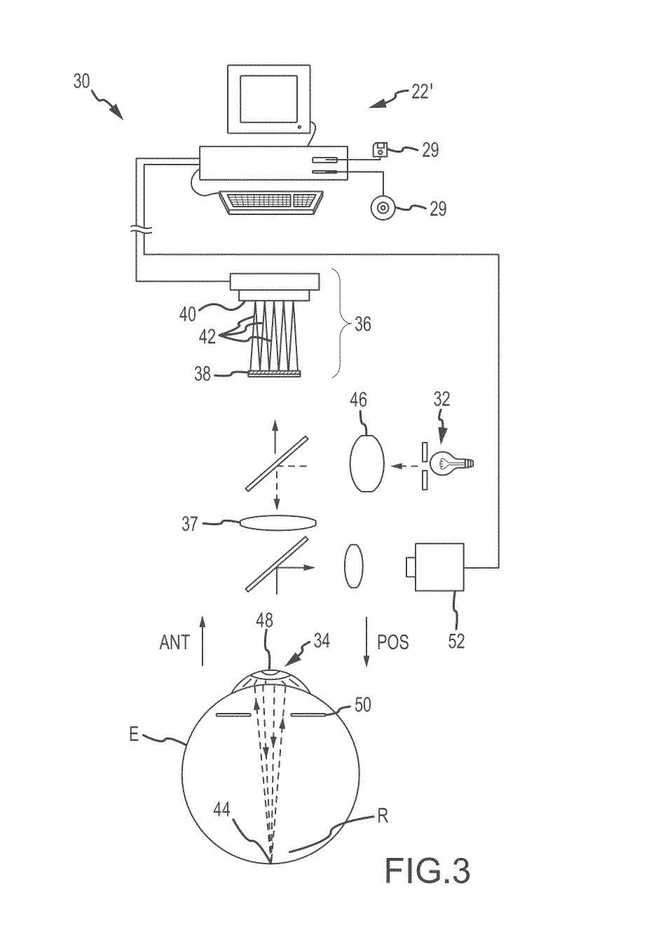

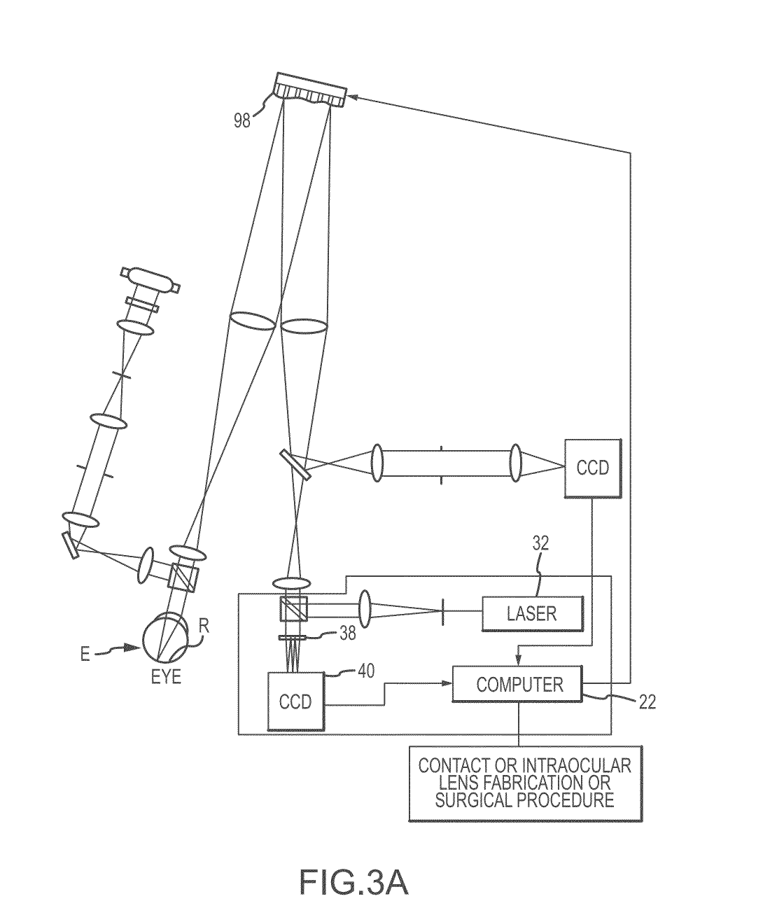

Referring now to FIG. 3, one embodiment of a wavefront measurement system 30 is schematically illustrated in simplified form. In very general terms, wavefront measurement system 30 is configured to sense local slopes of a gradient map exiting the patient's eye. Devices based on the Hartmann-Shack principle generally include a lenslet array to sample the gradient map uniformly over an aperture, which is typically the exit pupil of the eye. Thereafter, the local slopes of the gradient map are analyzed so as to reconstruct the wavefront surface or map.

More specifically, one wavefront measurement system 30 includes an image source 32, such as a laser, which projects a source image through optical tissues 34 of eye E so as to form an image 44 upon a surface of retina R. The image from retina R is transmitted by the optical system of the eye (e.g., optical tissues 34) and imaged onto a wavefront sensor 36 by system optics 37. The wavefront sensor 36 communicates signals to a computer system 22′ for measurement of the optical errors in the optical tissues 34 and/or determination of an optical tissue ablation treatment program. Computer 22′ may include the same or similar hardware as the computer system 22 illustrated in FIGS. 1 and 2. Computer system 22′ may be in communication with computer system 22 that directs the laser surgery system 10, or some or all of the components of computer system 22, 22′ of the wavefront measurement system 30 and laser surgery system 10 may be combined or separate. If desired, data from wavefront sensor 36 may be transmitted to a laser computer system 22 via tangible media 29, via an I/O port, via an networking connection 66 such as an intranet or the Internet, or the like.

Wavefront sensor 36 generally comprises a lenslet array 38 and an image sensor 40. As the image from retina R is transmitted through optical tissues 34 and imaged onto a surface of image sensor 40 and an image of the eye pupil P is similarly imaged onto a surface of lenslet array 38, the lenslet array separates the transmitted image into an array of beamlets 42, and (in combination with other optical components of the system) images the separated beamlets on the surface of sensor 40. Sensor 40 typically comprises a charged couple device or “CCD,” and senses the characteristics of these individual beamlets, which can be used to determine the characteristics of an associated region of optical tissues 34. In particular, where image 44 comprises a point or small spot of light, a location of the transmitted spot as imaged by a beamlet can directly indicate a local gradient of the associated region of optical tissue.

Eye E generally defines an anterior orientation ANT and a posterior orientation POS. Image source 32 generally projects an image in a posterior orientation through optical tissues 34 onto retina R as indicated in FIG. 3. Optical tissues 34 again transmit image 44 from the retina anteriorly toward wavefront sensor 36. Image 44 actually formed on retina R may be distorted by any imperfections in the eye's optical system when the image source is originally transmitted by optical tissues 34. Optionally, image source projection optics 46 may be configured or adapted to decrease any distortion of image 44.

In some embodiments, image source optics 46 may decrease lower order optical errors by compensating for spherical and/or cylindrical errors of optical tissues 34. Higher order optical errors of the optical tissues may also be compensated through the use of an adaptive optic element, such as a deformable mirror (described below). Use of an image source 32 selected to define a point or small spot at image 44 upon retina R may facilitate the analysis of the data provided by wavefront sensor 36. Distortion of image 44 may be limited by transmitting a source image through a central region 48 of optical tissues 34 which is smaller than a pupil 50, as the central portion of the pupil may be less prone to optical errors than the peripheral portion. Regardless of the particular image source structure, it will be generally be beneficial to have a well-defined and accurately formed image 44 on retina R.

In one embodiment, the wavefront data may be stored in a computer readable medium 29 or a memory of the wavefront sensor system 30 in two separate arrays containing the x and y wavefront gradient values obtained from image spot analysis of the Hartmann-Shack sensor images, plus the x and y pupil center offsets from the nominal center of the Hartmann-Shack lenslet array, as measured by the pupil camera 51 (FIG. 3) image. Such information contains all the available information on the wavefront error of the eye and is sufficient to reconstruct the wavefront or any portion of it. In such embodiments, there is no need to reprocess the

Hartmann-Shack image more than once, and the data space required to store the gradient array is not large. For example, to accommodate an image of a pupil with an 8 mm diameter, an array of a 20×20 size (i.e., 400 elements) is often sufficient. As can be appreciated, in other embodiments, the wavefront data may be stored in a memory of the wavefront sensor system in a single array or multiple arrays.

While the methods of the present invention will generally be described with reference to sensing of an image 44, a series of wavefront sensor data readings may be taken. For example, a time series of wavefront data readings may help to provide a more accurate overall determination of the ocular tissue aberrations. As the ocular tissues can vary in shape over a brief period of time, a plurality of temporally separated wavefront sensor measurements can avoid relying on a single snapshot of the optical characteristics as the basis for a refractive correcting procedure. Still further alternatives are also available, including taking wavefront sensor data of the eye with the eye in differing configurations, positions, and/or orientations. For example, a patient will often help maintain alignment of the eye with wavefront measurement system 30 by focusing on a fixation target, as described in U.S. Pat. No. 6,004,313, the full disclosure of which is incorporated herein by reference. By varying a position of the fixation target as described in that reference, optical characteristics of the eye may be determined while the eye accommodates or adapts to image a field of view at a varying distance and/or angles.

The location of the optical axis of the eye may be verified by reference to the data provided from a pupil camera 52. In the exemplary embodiment, a pupil camera 52 images pupil 50 so as to determine a position of the pupil for registration of the wavefront sensor data relative to the optical tissues.

An alternative embodiment of a wavefront measurement system is illustrated in FIG. 3A. The major components of the system of FIG. 3A are similar to those of FIG. 3. Additionally, FIG. 3A includes an adaptive optical element 53 in the form of a deformable mirror. The source image is reflected from deformable mirror 98 during transmission to retina R, and the deformable mirror is also along the optical path used to form the transmitted image between retina R and imaging sensor 40. Deformable mirror 98 can be controllably deformed by computer system 22 to limit distortion of the image formed on the retina or of subsequent images formed of the images formed on the retina, and may enhance the accuracy of the resultant wavefront data. The structure and use of the system of FIG. 3A are more fully described in U.S. Pat. No. 6,095,651, the full disclosure of which is incorporated herein by reference.

The components of an embodiment of a wavefront measurement system for measuring the eye and ablations may comprise elements of a WaveScan® system, available from VISX, INCORPORATED of Santa Clara, Calif. One embodiment includes a WaveScan system with a deformable mirror as described above. An alternate embodiment of a wavefront measuring system is described in U.S. Pat. No. 6,271,915, the full disclosure of which is incorporated herein by reference. It is appreciated that any wavefront aberrometer could be employed for use with the present invention. Relatedly, embodiments of the present invention encompass the implementation of any of a variety of optical instruments provided by WaveFront Sciences, Inc., including the COAS wavefront aberrometer, the ClearWave contact lens aberrometer, the CrystalWave IOL aberrometer, and the like.

### I. Pre-Treatment Refractive Measurement

When developing a treatment for a patient's vision condition, it is helpful to evaluate or consider the optical properties of the eye prior to the treatment. These optical properties can be used when determining a treatment for the patient's eye. Various measurement modalities can be used to assess the eye, including wavefront aberrometry, keratometry, topography, pupilometry, refractometry (e.g. measurement of manifest refraction), pachymetry, biometry, and the like. Such measurements are made prior to treating or retreating the eye.

In many current approaches, a subjective eye examination is performed so as to provide an initial measurement of a patient's low order aberrations. These subjective measurements can be taken using a standard phoropter, trial frames and lenses, or the like, and the manifest refraction identified during these measurements relies to a large extent on the patient's subjective evaluation of vision quality through a candidate corrective lens, most often by selection between alternative candidate lenses.

Once the standard sphere, cylindrical power, and cylinder angle have been identified, current approaches may optionally include a wavefront examination, with the wavefront measurements specifically being performed to provide an additional assessment of the patient's eye. The wavefront measurement may utilize the results of the standard, subjective manifest measurements. For example, many wavefront aberrometers include optical elements which adjust or precompensate for standard optical errors of the eye being measured so as to more accurately and reliably measure the high-order aberrations, for example, by avoiding cross-over between local gradient-indicating spots from one element of the Hartmann-Shack lenslet array being misinterpreted as being associated with another element of the array. Before finalizing a treatment based on the wavefront examination, subjective manifest refraction examination may again be separately performed.

Notwithstanding any use of a subjectively-measured manifest refraction in obtaining an instrument-based objective measurement of the eye, embodiments of the present invention encompass techniques wherein the treatment is based only upon the results of an objective optical manifest refraction, which can be determined for example based on a wavefront examination. In some cases, the results of the objective optical manifest refraction examination override the results of any measured subjective manifest refraction. Optionally, a prescription can be determined without measuring the subjective manifest refraction. Thus, even where there is a significant difference between a subjective manifest refraction and an objective optical manifest refraction, such as a 0.2 or 0.3 sphere difference, the objective optical manifest refraction can be used to determine the treatment, and subjective manifest refraction can be disregarded.

In some cases, subjective manifest refraction results can also be used to develop a filter for a wavefront examination procedure. Wavefront analysis typically captures both low order and high order aberrations, whereas subjective manifest refraction captures low order aberrations. In some instances, the presence of low order aberrations can limit the ability of a wavefront mechanism to accurately evaluate the high order aberrations. Using the results of a subjective manifest refraction examination, however, it is possible to develop a low order filter. The filter may include, for example, a set of one or more lenses that are placed between the wavefront mechanism and the patient's eye. The filter operates to cancel out or counteract the low order aberrations (e.g. cylinder), and thus only information corresponding to the high order aberrations reaches the wavefront mechanism. Optionally, a filter can be implemented by software or hardware modules of a treatment system. For example, a subjective manifest refraction measurement can be inputted into a treatment system, which in turn compensates for low order aberrations present in the subjective manifest when performing a wavefront examination or evaluating wavefront examination results.

Hence, subjective manifest refraction measurements can be performed in order to establish a filter or cancelation factor or mechanism for a wavefront examination, to establish a baseline or starting point for a wavefront examination, or to establish an adjustment factor or mechanism for a wavefront examination. Embodiments of the present invention encompass methods in which a subjective manifest refraction is not performed at all, or in which a subjective manifest refraction is performed but not used in combination with a wavefront examination when adjusting or generating a prescription for a patient's eye. A measured subjective manifest refraction may, however, be used as a basis for, or to facilitate, a wavefront evaluation.

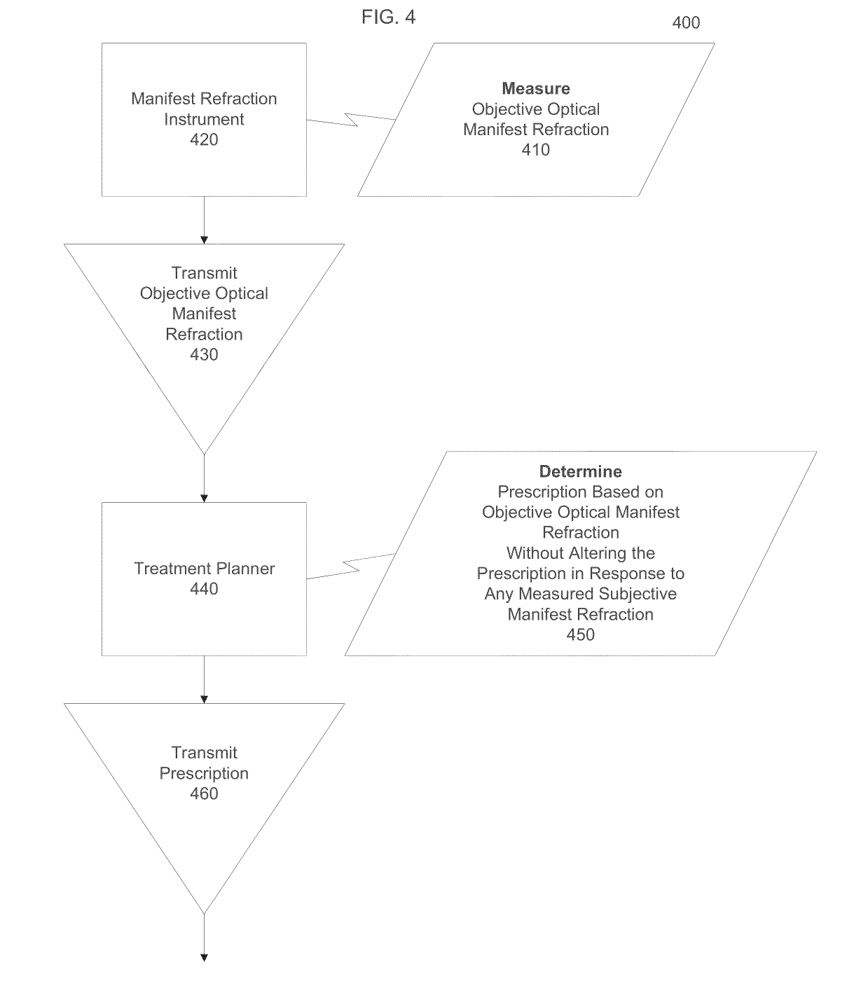

FIG. 4 shows aspects of an exemplary method 400 for treating an eye of a patient. Method 400 includes measuring an objective optical manifest refraction of the patient's eye, as indicated by step 410. Measurement of the manifest refraction can be performed with a manifest refraction instrument 420. The manifest refraction instrument can be an apparatus such as a wavefront measurement assembly, a combined wavefront and topography measurement assembly, an aberrometer assembly, and the like. The method also includes transmitting objective optical manifest refraction measurement data, as indicated by step 430, from the manifest refraction instrument to a treatment planner 440. The treatment method further includes determining a prescription for the patient's eye based on the objective optical manifest refraction, without altering the prescription in response to any measured subjective manifest refraction, as indicated by step 450. Determination of the prescription can be performed with treatment planner 440. Method 400 additionally includes transmitting the prescription from the treatment planner, as indicated by step 460, so as to facilitate surgically altering the eye per the prescription. In some instances, the manifest refraction instrument and the treatment planner can be configured as components of a single system that can perform measurement, planning, and treatment procedures, for example.

### A. Refractometry Measurement (Objective Manifest)

The objective optical manifest refraction measurement can be based on or determined by a wavefront evaluation of the eye. U.S. Pat. Nos. 6,808,266 and 7,029,119 describe approaches for objectively obtaining a manifest refraction value of a patient's eye based on certain Zernike wavefront aberration data. Relatedly, U.S. Pat. Nos. 6,761,454, 7,114,808, 7,461,938, and 7,490,940 describe techniques for determining sphere and cylinder components of subjective refraction using an objective wavefront measurement. Additionally, techniques for predicting refraction from wavefront aberrations are discussed in Thibos, “Accuracy And Precision Of Objective Refraction From Wavefront Aberrations” J. Vision 4:329-351 (2004).

Typically, wavefront examination involves passing a wave of light into the patient's eye, and analyzing the quality of light which is reflected back out of the patient's eye. In this way, it is possible to analyze the optical properties of the patient's visual based on how the eye transforms or alters the light wave. Wavefront examination is particularly useful in diagnosing vision conditions or characterizing vision performance in an eye of a patient. In some cases, the objective optical manifest refraction measurement can be based on or determined by a wavefront evaluation of the eye. In some cases, the objective optical manifest refraction measurement can be based on or determined by a combined wavefront and topographic evaluation of the eye. Topographic examination is typically used to evaluate the surface curvature or shape of the patient's cornea, which can play a significant role in the focusing ability of the eye. Hence, wavefront techniques, optionally in combination with corneal topography, can provide an objective optical basis for evaluating the optical properties of the patient's eye. In some cases, wavefront evaluation data may be used to determine a vision treatment for a patient. In some cases, wavefront evaluation data in combination with topographic evaluation data can be used to determine a vision treatment for a patient. Hence, wavefront techniques, optionally in combination with corneal topography, can provide an objective optical basis for determining a vision treatment for an eye of a patient. It has been discovered that an objective optical manifest refraction measurement can be used solely or primarily to derive a prescription for a patient.

### B. Refractometry Measurement (Subjective Manifest)

Exemplary vision treatment techniques may include measuring the subjective manifest refraction of the eye, interactively in response to subjective input from the patient. For example, the subjective manifest refraction measurement can be based on or determined by a phoropter evaluation of the eye. Phoropter mechanisms typically include a series of lenses which focus or refract light into the patient's eye, thus allowing the operator to calculate how much sphere, cylinder, and axis is needed to treat the patient's refractive error. In some cases, the subjective manifest refraction measurement can be based on or determined by a trial frame evaluation of the eye. Optionally, the subjective manifest refraction measurement can be based on or determined by a trial lens evaluation of the eye.

In some cases, subjective manifest can be measured manually. Optionally, subjective manifest can be measured using a computer. Typically, measurement of subjective manifest takes into account the effect of patient neural signals or processing, and may reflect particular aspects of the patient's perception of vision, what the patient is accustomed to seeing, or specific vision preferences of the patient. The subjective measurement of manifest refraction therefore may present some level of uncertainty or unpredictability, for example with regard to how light is received at the patient's retina, how nerve signals corresponding to that light are transmitted to the brain via the optic nerve, and how the signals are perceived or processed by neural tissue such as the brain.

Optionally, subjective manifest can be measured with an autorefractor. In some instances, subjective manifest can be determined based on a combined manual and autorefractor evaluation of the eye. For example, an operator may use an autorefractor to determine a baseline refraction of the eye, and a manual phoropter to determine the actual prescription, taking into account the results of the autorefractor examination.

The subjective manifest refraction measurement may differ significantly from the optical manifest refraction measurement. For example, the objective optical manifest refraction measurement may differ from the subjective manifest refraction measurement by more than about 0.10 Diopters. In some cases, the objective optical manifest refraction measurement may differ from the subjective manifest refraction measurement by more than about 0.25 Diopters. Similarly, the objective optical manifest refraction measurement may differ from the subjective manifest refraction measurement by more than about 0.37 Diopters. Relatedly, the objective optical manifest refraction measurement may differ from the subjective manifest refraction measurement by more than about 0.50 Diopters. Despite such significant differences between the objective optical manifest refraction measurement and the subjective manifest refraction measurement, a prescription for the patient's eye can be determined effectively based on the objective optical manifest refraction, without altering the prescription in response to the measured subjective manifest refraction. In some cases, techniques may involve remeasuring the objective manifest, the subjective manifest, or both, when there is a significant difference between the measured objective and subjective manifest refractions.

According to some embodiments, the eye can be surgically altered under direction of a treating physician, such that the eye is surgically altered without the treating physician measuring subjective manifest refraction of the eye or otherwise obtaining the subjective manifest refraction of the eye in preparation for the surgical alteration.

Although subjective manifest measurements can be used as a safety check, to establish a filter, or to establish a baseline for objective optical measurements, it is possible to ignore or disregard the subjective manifest when developing a prescription for treatment purposes.

By determining a prescription or treatment for a patient based on an objective optical manifest refraction as measured by an objective instrument, without altering the prescription or treatment based on a subjective manifest refraction measurement as measured by a subjective manual procedure, it is possible to directly transmit objective optical manifest refraction measurement data from the objective instrument directly to a treatment planner. Hence, the prescription or treatment can be generated without manually measuring the subjective manifest, or manually entering the subjective manifest into a planner, or both.

In contrast to a subjective manifest refraction approach, objective manifest refraction embodiments of the present invention involve an optical technique that does not involve the uncertain or unpredictable aspects of the subjective manifest. Hence, the optical approach can provide enhanced accuracy when preparing a prescription for the patient.

### C. Objective Manifest with Subjective Aspects

In some instances, objective optical manifest refraction measurements may include techniques having a subjective or neural adaptation component that are not related to phoropters or other traditional subjective manifest refraction devices. For example, objective optical manifest refraction measurements can be based on results obtained from a device having deformable mirror optics, such as an adaptive optics system. Results from such wavefront or objective measurements may reflect a subjective or patient-input based element. In some cases, an adaptive optics system or similar vision simulator may be used to provide the patient with a preview of the effect a particular vision treatment (e.g. negating or diminishing aberrations) may have on their vision.

### D. Manifest Parameters (Sphere)

Manifest refraction can include a sphere component. Hence, a objective optical manifest refraction can include an objective optical sphere value, and a subjective manifest refraction can include a subjective sphere value. Exemplary techniques can include determining a prescription for the patient's eye based on an objective optical manifest sphere value, without altering the prescription in response to any measured subjective manifest refraction sphere value. The objective sphere value may differ from the subjective sphere value by more than about 0.3 Diopters. In some cases, the difference may be between about 0.5 Diopters and about 1.0 Diopters. In some cases, the difference may be greater than about 0.5 Diopters. Despite such significant differences between the objective sphere measurement and the subjective sphere measurement, a prescription for the patient's eye can be determined effectively based on the objective sphere value, without altering the prescription in response to the measured subjective sphere value.

Hence, embodiments encompass techniques for determining a prescription where the results from an objective optical manifest examination override any results from a subjective manifest examination. Relatedly, embodiments encompass approaches for determining a prescription based upon the results from an objective optical manifest examination, in the absence of performing a subjective manifest examination. Systems for performing such techniques may include a manifest refraction instrument in cooperative association with a treatment planner. The manifest refraction instrument can operate to measure an objective optical manifest refraction of the eye of the patient, and the treatment planner can operate to determine a prescription for the patient, based on the objective optical manifest refraction measurement. The prescription can be used to facilitate a surgical alteration of the eye. The system may include an input for receiving a subjective manifest refraction of the eye, which can be used as a safety check or low order filter as described elsewhere herein. Optionally, the system may be configured to receive the subject manifest refraction, but not to use the subjective manifest when determining a prescription. In some instances, the system may have no input that receives a subjective manifest refraction.

### II. Refractive Treatment Planning

Embodiments of the present invention encompass any of a variety of pre-treatment refractive measurements that can be used to for planning a desired refractive treatment for the patient. For example, embodiments may include aspects of measurement and planning techniques such as those described in U.S. patent application Ser. Nos. 12/418,841 filed Apr. 6, 2009, and 61/428,644 filed Dec. 30, 2010, the contents of which are incorporated herein by reference.

Exemplary treatment planners can use objective optical manifest refraction measurement data obtained from a manifest refraction instrument to generate a prescription or treatment for the patient. Such prescriptions can be determined without alteration in response to any subjective manifest refraction measurement of the eye.

### A. Variability in Subjective Manifest Refraction Measurements

Studies have shown the existence of significant clinician variability in measurements of subjective refraction. For example, with regard to the repeatability of clinician refraction, a mean spherical equivalent between two clinicians was observed to have a distribution of −0.12 Diopters with a standard deviation of 0.41 Diopters. In some cases, clinical variability in subjective refraction may be due to inconsistencies associated with the patient's visual perception. In some cases, the standard deviation may be within a range from about 0.25 Diopters to about 0.50 Diopters.

### B. Accuracy in Objective Manifest Refraction Measurements

Embodiments of the present invention provide for objective manifest refraction measurements having a standard deviation of about 0.15 Diopters. Hence, the instant techniques provide for the measurement of objective optical manifest refraction, to standard deviations significantly less than the 0.37 Diopter standard deviation associated with a subjective manifest refraction measurement of the eye. In some cases, the standard deviation may be within a range from about 0.25 Diopters to about 0.50 Diopters. These objective measurements are much more repeatable and accurate than the currently used subjective manifest refraction measurements obtained with phoropters or the like. Contrary to current practices in prescription development, which often are based in large part upon incorporating the results of phoropter evaluations and other subjective manifest refraction measurements, the techniques disclosed herein are well suited for use in generating prescriptions without relying upon any subjective manifest refraction measurements.

It is possible to achieve such accurate objective measurements by using high resolution wavefront systems having dense arrays, with short focal lengths that provide no aliasing. Exemplary high resolution wavefront systems and techniques are discussed by Neal et al. in “Effect of lenslet resolution on the accuracy of ocular wavefront measurements” Proc. SPIE 4245, 78-91 (2001), “Shack-Hartmann wavefront sensor precision and accuracy” Proc. SPIE 4779, 148-160 (2002), and U.S. Pat. Nos. 6,550,917 and 6,634,750, the contents of which are incorporated herein by reference. These accurate objective measurements can allow for the development of more precise treatments. For example, higher confidence in the accuracy of the measurement allows the operator or physician to plan and administer more aggressive prescription shapes based on those measurements. In some instances, such treatments may include contract lens prescriptions, scleral lens prescriptions, laser sculpting or treatment prescriptions, IOL prescriptions including custom IOL prescriptions, and spectacle prescriptions including prescriptions that involve different pairs of glasses for different uses (e.g. low ambient lighting conditions, high ambient lighting conditions).

As noted elsewhere herein, although the subjective manifest refraction measurement may differs from the optical manifest refraction measurement, exemplary techniques involve developing a prescription for the patient based on the objective optical manifest refraction, without altering the prescription in response to the measured subjective manifest refraction. Such approaches can be implemented even where there is a significant difference, for example 0.37 Diopters or more, between the subjective and objective optical manifests. Likewise, such approaches can be implemented even where the objective measurement differs from the subjective measurement by more than a standard deviation associated with subjective measurement. Hence, instead of generating and applying a prescription that reflects the patient's neural processing for vision, a prescription is based on optical factors alone. In some instances, the difference between the subjective and objective optical manifests can be more than about 0.40 or 0.50 Diopters. The ability to generate effective prescriptions in such circumstances by disregarding the subjective manifest, or by determining the prescription independently of the subjective manifest, is surprising and unexpected.

In some cases, techniques may involve repeating an objective measurement, a subjective measurement, or both, when there is a significant difference between original objective and subjective measurements. Relatedly, significant differences between objective and subjective measurements (e.g. 0.50 Diopters) can be used as part of a safety check or a reference, where a decision is made whether to proceed with a treatment based on an objective measurement.

In some cases, objective and subjective results can be compared, for example, by providing a patient with an objective-based treatment in one eye, and a subjective-based treatment in the other eye. In some cases, objective-based treatments and subjective-based treatments can be compared using adaptive optics or similar vision simulation techniques.

### B. Visual Acuity and Contrast Sensitivity

In some cases, it is possible to evaluate the difference between prescriptions based on objective and subjective manifest refraction measurements, without administering the prescription to the patient in a surgical procedure. For example, the visual acuity can be determined based on objective and subjective manifest refraction measurements, without surgically treating the patient. A wavefront based refraction that provides a better visual acuity can be considered as more accurate than a phoropter based refraction that provides a worse visual acuity.

In some instances, the accuracy or precision of the objective manifest refraction measurement can be evaluated based on visual acuity or contrast sensitivity techniques. Likewise, the accuracy or precision of an objective measurement approach can be compared with the accuracy or precision of a subjective measurement approach.

### C. Adaptive Optics

Adaptive optic techniques can be used to evaluate the difference between an objective-based treatment (e.g prescription determined by wavefront measurement) and a subjective-based treatment (e.g. prescription determined by phoropter). In some instances, adaptive technique evaluations can be performed after the patient treatment is allowed to stabilize. Embodiments of the present invention therefore encompass techniques that involve the use of adaptive optics for diagnostic applications as well as development applications.

### D. Cylinder

Cylinder can be used as a prescription measure for astigmatism, and is a low order aberration. In some cases, currently used techniques may involve undercorrecting or undermeasuring cylinder. It has been reported that the use of conventional manifest refraction in excimer laser vision treatment can result in an undercorrection of cylinder. For example, Choi et al., “Excimer Laser Photorefractive Keratectomy for Astigmatism” Korean J. Ophthalmol., Vol. 7:20-24 (1993) reports a mean refractive cylinder change from 1.62 Diopters to 0.48 Diopters following a manifest-guided ablation procedure. In contrast, by using objective manual manifest refraction measurements, it is possible to compensate for the full cylinder component of the vision measurement, and correcting for all of the cylinder on the cornea can lead to a better treatment outcome. Hence, objective optical manifest refraction techniques are well suited for correcting for cylinder. In some cases, exemplary techniques may include fully measuring cylinder (e.g. with little or no under-measurement) and fully correcting cylinder (e.g. with little or no under-correction). Such techniques can be applied even where a patient may already be accustomed to, or otherwise express an initial preference for, an under-correction for cylinder. It has been observed that autorefractors may be particularly effective in obtaining cylinder measurements (see e.g. Cheng et al. “Predicting subjective judgment of best focus with objective image quality metrics” Journal of Vision, 4, 310-321 (2004), the content of which is incorporated herein by reference.

### E. Registration and Combination

Embodiments of the present invention encompass techniques for registering or combining objective optical manifest refraction measurements with other measurements provided by pupillometry, aberrometry, topography, and other evaluation modalities for the development of treatment prescriptions. Such evaluation modalities can reside on a single instrument or system, and can be registered to each other, for example as described in U.S. patent application Ser. No. 12/418,841 filed Apr. 6, 2009 (Atty. Docket No. 18158C-037410US).

### F. Refraction Study Results

### 1. 4 mm Sphere Correlation to Manifest Refraction

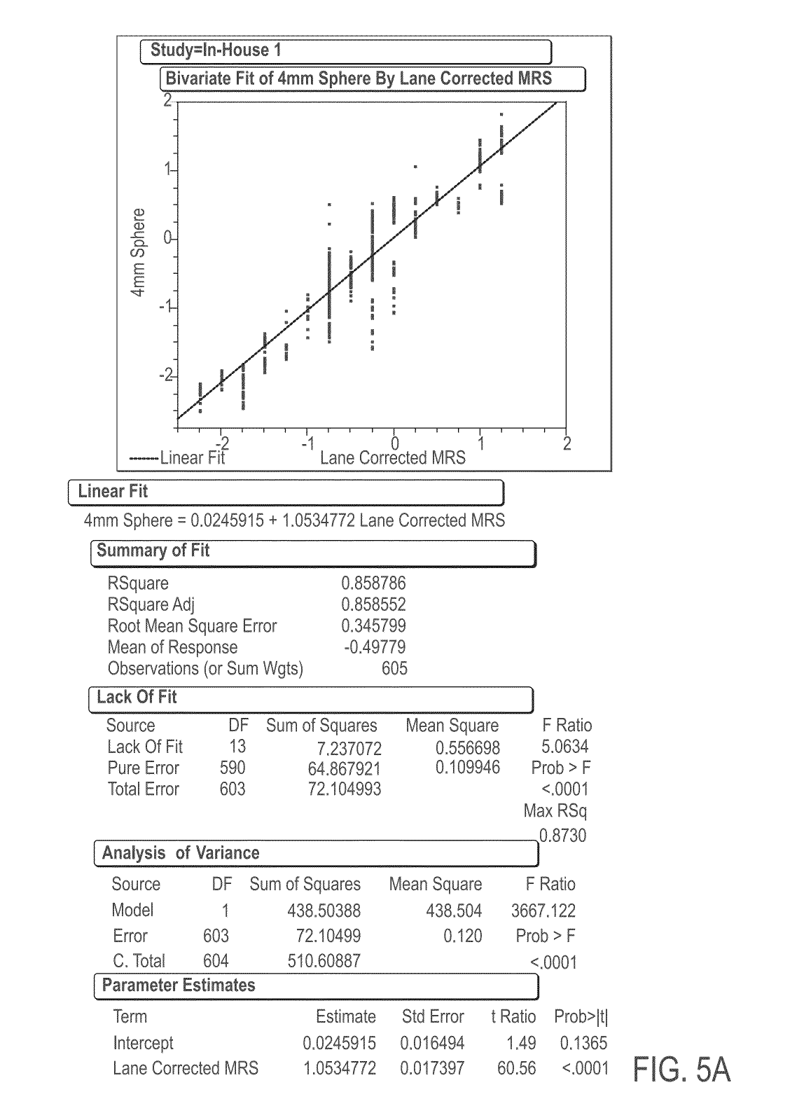

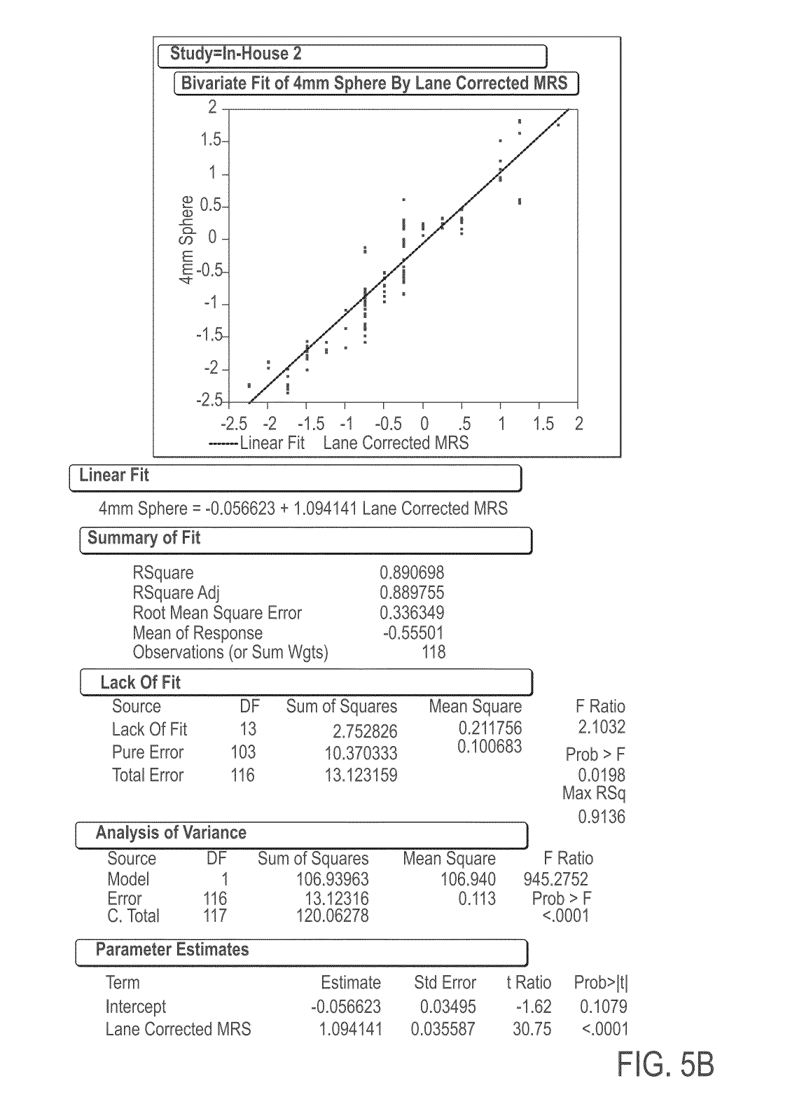

As depicted in FIGS. 5A and 5B, the root mean square (RMS) error in fit is dominated by uncertainty in the manifest refraction (RMS=0.34 D; Slope=1.09; Intercept=−0.06 D; R²=0.89). A wider range of manifest refraction (MR) may be useful in a field study. Three or more manifest refraction measurements per eye may be useful to mitigate MR uncertainty. More than three sphere measurements per eye may be helpful for a field study.

### 2. 4 mm Sphere Repeatability

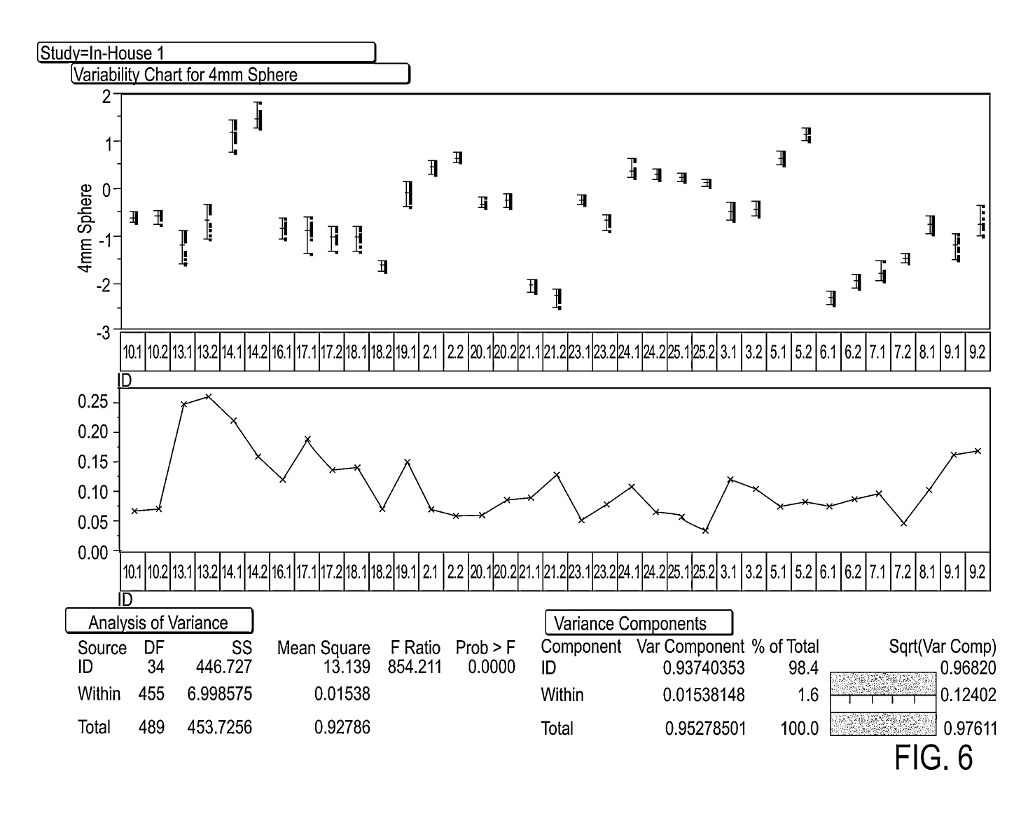

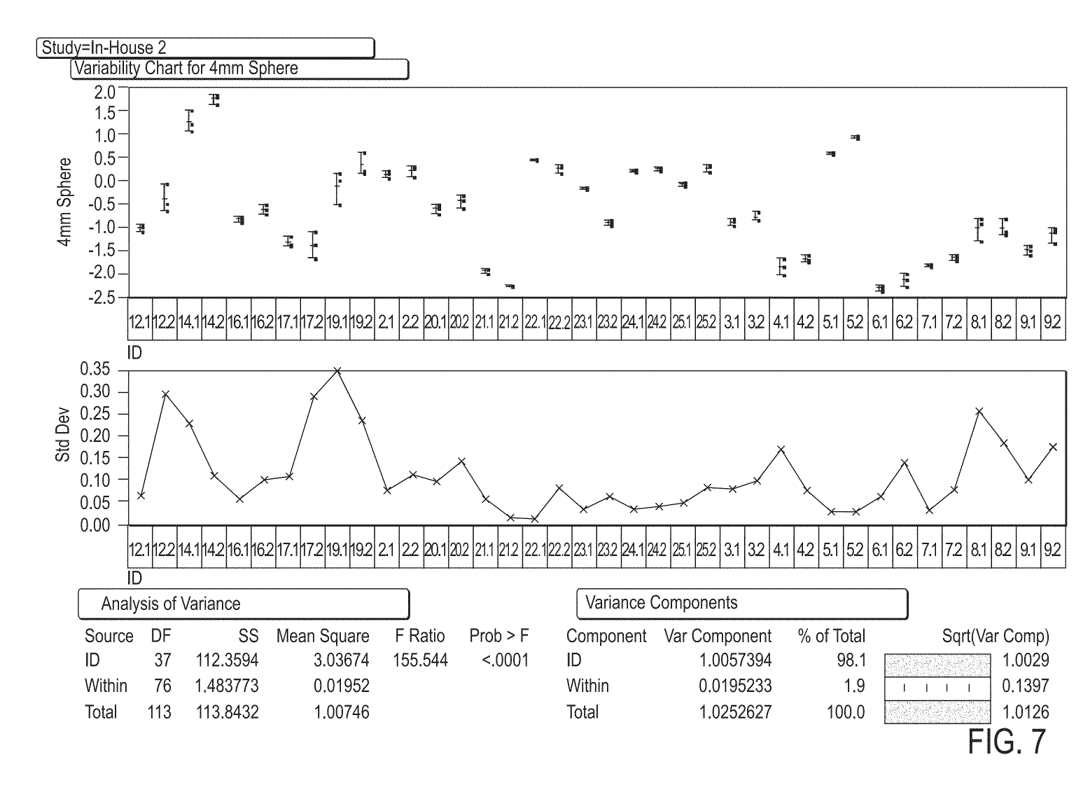

FIG. 6 shows the results of a first study involving thirty five eyes, where fourteen measurements per eye were performed. All eyes with fourteen or more measurements were included. The sphere repeatability was observed to be about 0.12 Diopters. FIG. 7 shows the results of a second study involving thirty eight eyes, where three measurements per eye were performed. All eyes with three or more measurements were included. The sphere repeatability was observed to be about 0.14 Diopters.

### 3. 4 mm Sphere Variability and Age

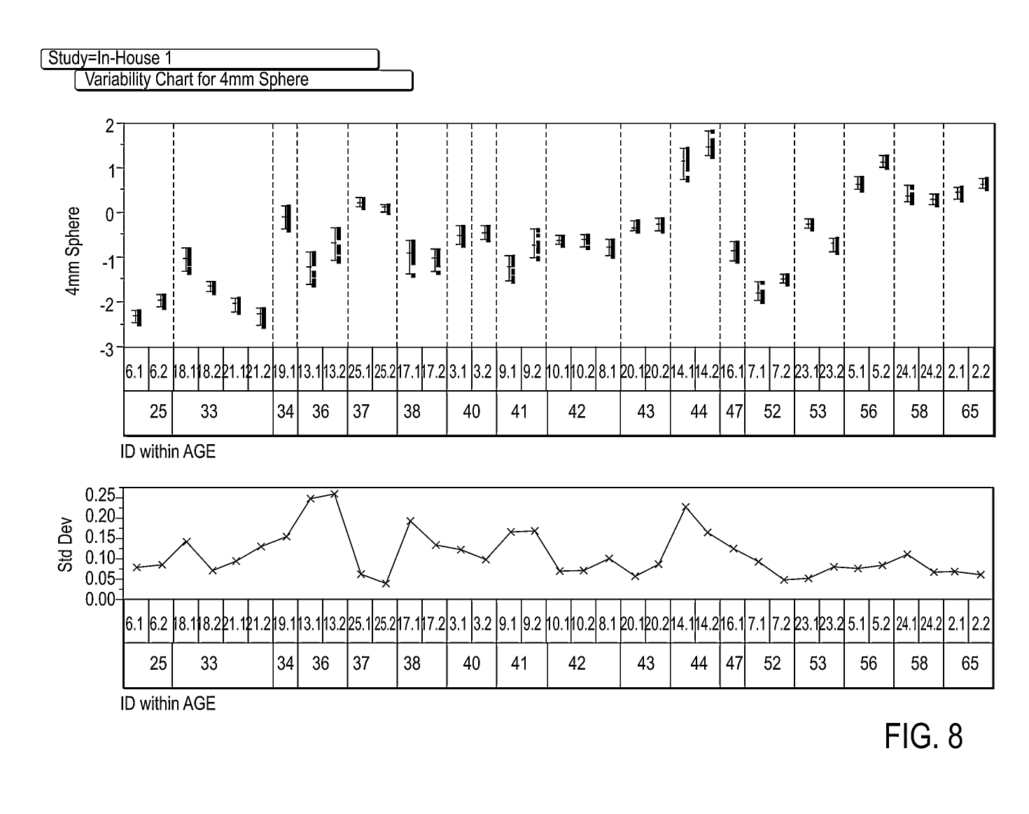

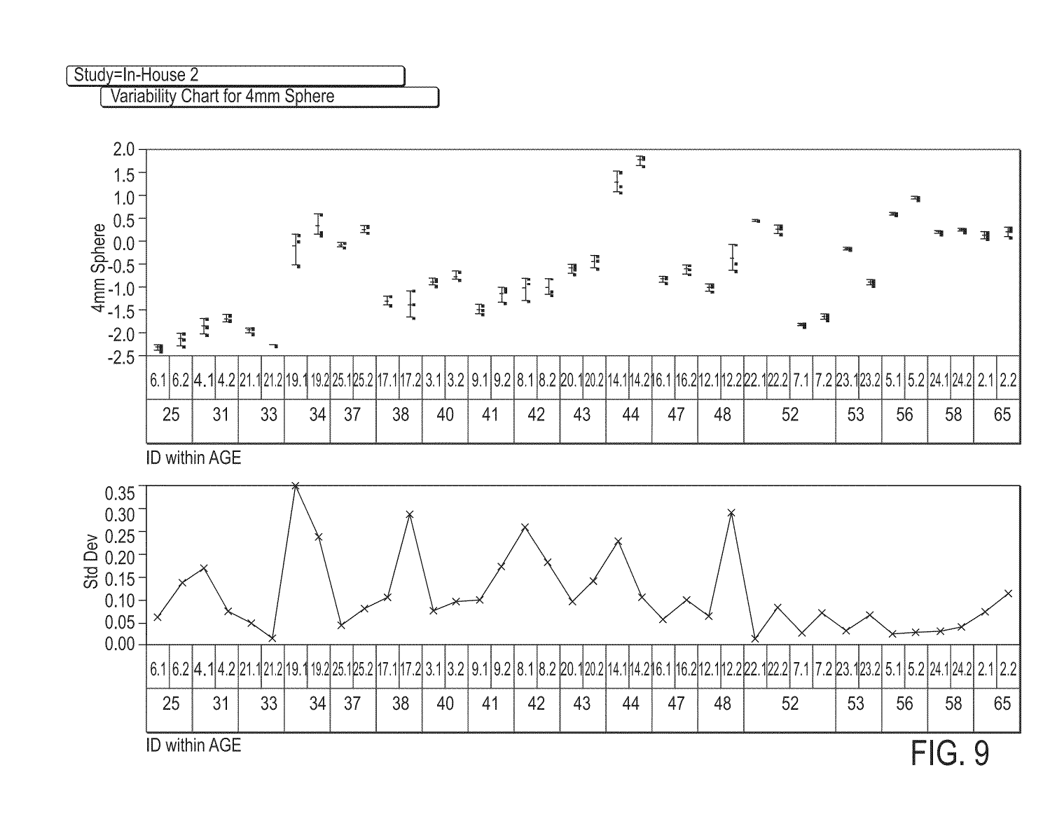

FIG. 8 provides the results of a first study showing that sphere variability is greater for younger patients. As depicted in the lower panel, the repeatability is 0.12 Diopters and there is a slight trend with age. FIG. 9 provides the results of a second study also showing that sphere variability is greater for younger patients. As depicted in the lower panel, the repeatability is 0.14 Diopters and there is a slight trend with age.

### 4. 4 mm Cylinder Repeatability

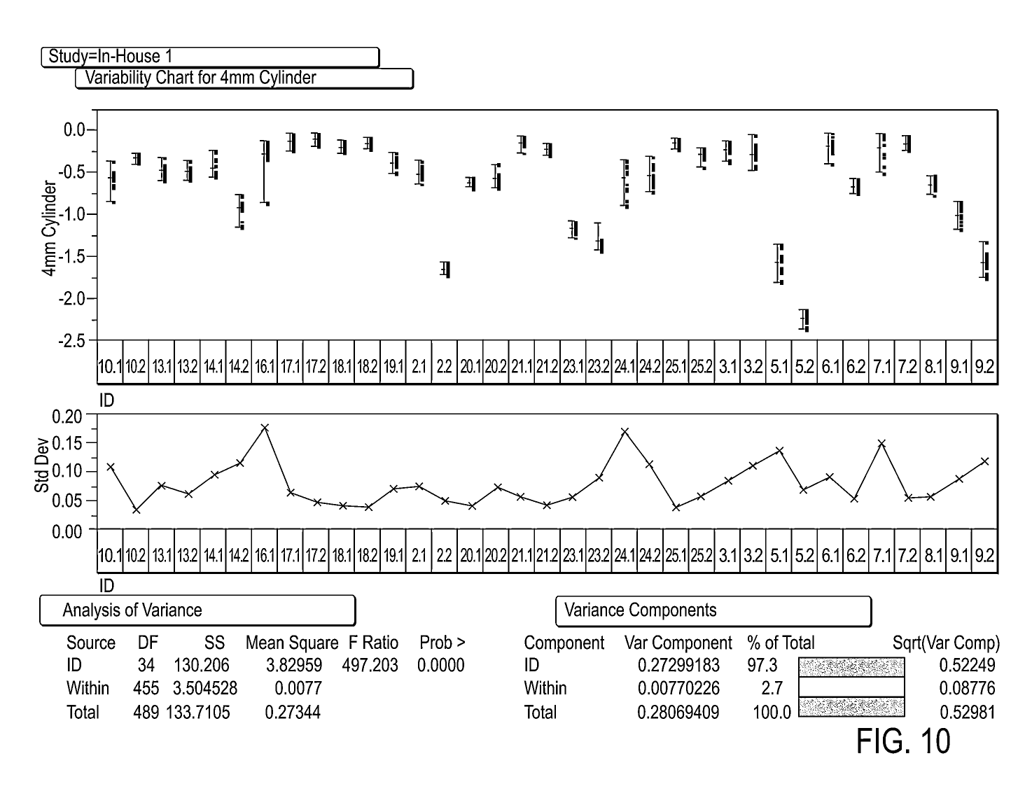

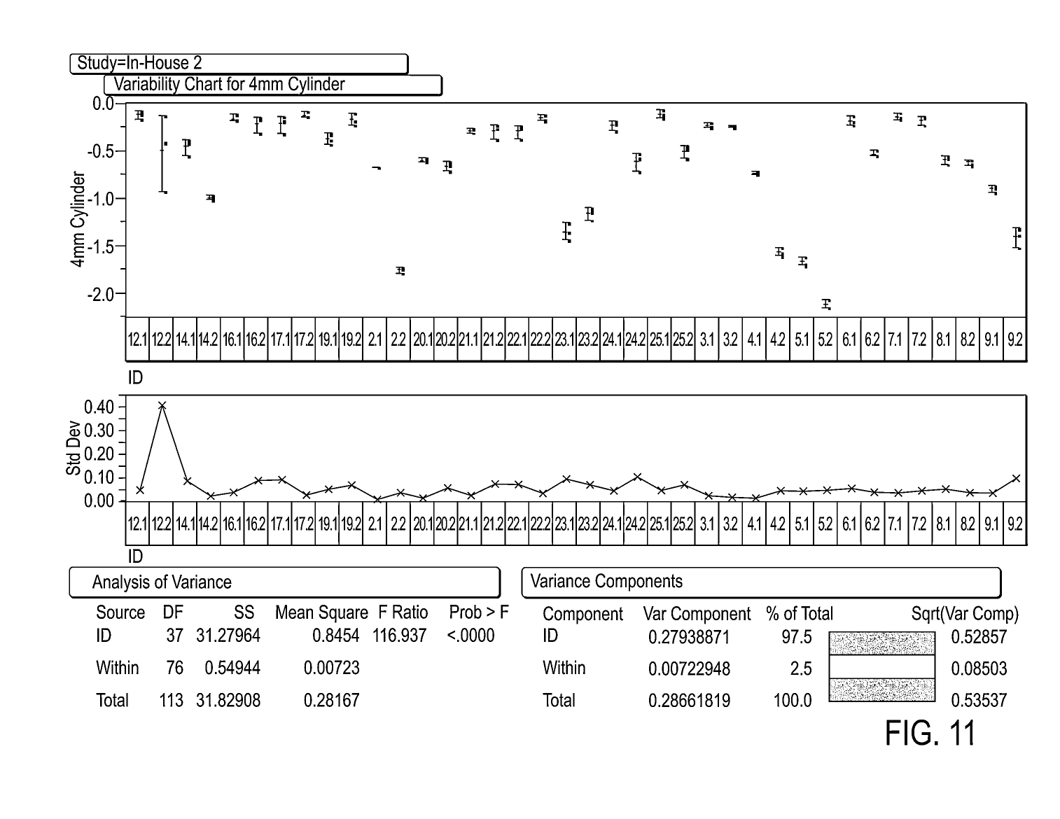

FIG. 10 shows the results of a first study, where cylinder repeatability was observed to be about 0.088 Diopters. FIG. 11 shows the results of a second study, where cylinder repeatability was observed to be about 0.085 Diopters. The cylinder repeatabilities of the first and second studies are similar.

### 5. 4 mm Sphere and Cylinder Correlation to Manifest Refraction

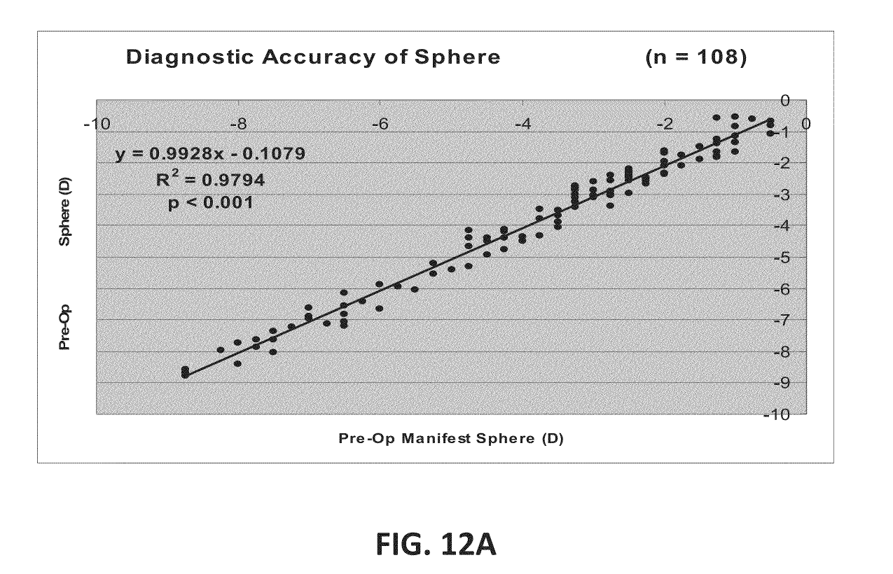

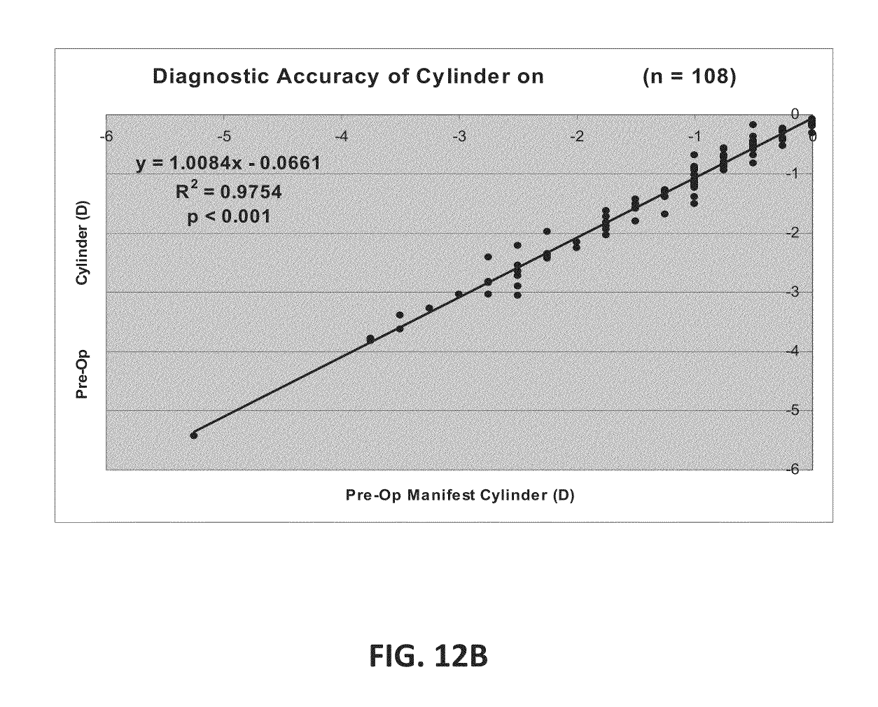

FIGS. 12A and 12B, depict correlation between subjective (e.g. Manifest Refraction) and objective (e.g. wavefront-derived) measurement methods for a group of 108 pre-operative eyes enrolled in a clinical study. FIG. 12A shows pre-operative manifest refraction versus pre-operative objective refraction for sphere. The comparison characterizes an ability to accurately measure the optics of the eye, when using either wavefront-derived values of sphere or those obtained by a manifest refraction method. Refractions were calculated over a 4 mm pupil at 12.5 mm vertex. FIG. 12B shows pre-operative manifest refraction versus pre-operative objective refraction for cylinder. The comparison characterizes an ability to accurately measure the optics of the eye, when using either wavefront-derived values of cylinder or those obtained by a manifest refraction method. Refractions were calculated over a 4 mm pupil at 12.5 mm vertex. The data shows a strong correlation (R-square of 0.9794 and 0.9754, respectively) between the manifest measurement and the wavefront-derived measurement of sphere and cylinder for the same eye.

In the studies represented by FIGS. 5A to 12B, some corrections may have been applied to the data. Spherical equivalent (SEQ) and Cylinder results are from Study 1 which was corrected. One correction was for Lane Length to the MRS. Also, the correct assignment of 4 fliers and one measurement where the subject later admitted to intentionally not comply.

This data supports the finding that repeatability of aberrometers is better than manifest refraction across the study populations. It may be noted that later studies concentrated on increased pupil size at the slight expense of sphere precision. In practice, it may be desirable to separate patients who accommodate from patients who do not (e.g. presbyopes). Repeatability with presbyopic patients is believed to correlate with instrument repeatability. Measurements on presbyopes allow the issue of chromatic correction to be precisely addressed. Young patients can be assessed separately. An asymmetric distribution skewed to the myopic side may be expected. It is helpful to avoid accommodation during the measurement process prior to the development and administration of a prescription treatment, particularly in young patients. Relatedly, it is helpful to ensure that an appropriate or maximum amount of information from one or more measurements is used to set the autorefraction level. For example, it may be useful to not autorefract to a significantly more myopic setting than was previously measured. Further, it may be desirable to dynamically track a sphere reading and provide feedback to operator, a patient, or both. Moreover, it may be helpful to allow a patient to participate more actively in the measurement, for example, by gating the measurement when ready, triggering measurement, providing a placebo button, and the like. Also, it may be useful to evaluate dynamically changing fixation targets.

The methods and apparatuses of the present invention may be provided in one or more kits for such use. The kits may comprise a system for profiling an optical surface, such as an optical surface of an eye, and instructions for use. Optionally, such kits may further include any of the other system components described in relation to the present invention and any other materials or items relevant to the present invention. The instructions for use can set forth any of the methods as described herein.

Each of the calculations or operations described herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like.

All patents, patent publications, patent applications, journal articles, books, technical references, and the like discussed in the instant disclosure are incorporated herein by reference in their entirety for all purposes.

While the above provides a full and complete disclosure of the preferred embodiments of the present invention, various modifications, alternate constructions and equivalents may be employed as desired. Therefore, the above description and illustrations should not be construed as limiting the invention, which can be defined by the claims.

1. A method for treating an eye of a patient, the method comprising:
measuring, with a manifest refraction instrument, an objective optical manifest refraction of the eye of the patient;
transmitting the objective optical manifest refraction measurement from the manifest refraction instrument to a treatment planner;
determining, with the treatment planner, a prescription based on the objective optical manifest refraction measurement, without altering the prescription in response to any subjective manifest refraction measurement of the eye; and
transmitting the prescription from the treatment planner so as to facilitate surgically altering the eye per the prescription.

2. The method according to claim 1, wherein the objective optical manifest refraction measurement comprises a wavefront evaluation of the eye.

3. The method according to claim 1, wherein the objective optical manifest refraction measurement comprises a combined wavefront and topographic evaluation of the eye.

4. The method according to claim 1, further comprising interactively, in response to subjective input from the patient, measuring the subjective manifest refraction of the eye, the subjective manifest refraction measurement differing significantly from the optical manifest refraction measurement.

5.-7. (canceled)

8. The method according to claim 4, wherein the objective optical manifest refraction measurement transmitted from the manifest refraction instrument differs from the subjective manifest refraction measurement by more than about 0.50 Diopters.

9. The method according to claim 4, wherein the subjective manifest refraction measurement comprises a phoropter evaluation of the eye.

10. The method according to claim 4, wherein the subjective manifest refraction measurement comprises a trial frame evaluation of the eye.

11. The method according to claim 4, wherein the subjective manifest refraction measurement comprises a trial lens evaluation of the eye.

12. The method according to claim 1, wherein the objective optical manifest refraction comprises an objective optical sphere value, and the subjective manifest refraction comprises a subjective sphere value that differs from the optical sphere value by greater than about 0.3 Diopters, the method further comprising:
repeating at least one of the subjective manifest refraction measurement and the objective manifest refraction measurement in response to the difference in objective optical and subjective sphere values; and
determining and imposing the prescription on the eye without altering the objective optical manifest refraction or the transmitted prescription in response to the subjective manifest refraction measurement(s).

13. The method according to claim 1, wherein the eye is surgically altered under direction of a treating physician, and wherein the eye is surgically altered without the treating physician measuring subjective manifest refraction of the eye or otherwise obtaining the subjective manifest refraction of the eye in preparation for the surgical alteration.

14.-15. (canceled)

16. A method for treating an eye of a patient, the method comprising:
receiving an objective optical manifest refraction of the eye of the patient;
transmitting the objective optical manifest refraction measurement from the manifest refraction instrument to a treatment planner;
determining, with the treatment planner, a prescription based on the objective optical manifest refraction measurement, without altering the prescription in response to any subjective manifest refraction measurement of the eye; and
transmitting the prescription from the treatment planner so as to facilitate surgically altering the eye per the prescription.

17. The method according to claim 16, further comprising measuring the objective optical manifest refraction of the eye of the patient.

18.-26. (canceled)

27. A system for deriving a prescription for an eye of a patient, the system comprising:
a wavefront-based instrument that measures a wavefront-derived objective optical manifest refraction measurement of the eye of the patient, the wavefront-derived manifest refraction comprising a sphere component, a cylinder component, and an axis component;
a treatment planner that determines a prescription based on the objective optical manifest refraction measurement, without altering the prescription in response to any subjective manifest refraction measurement of the eye; and
a surgical device that alters the eye per the prescription.

28. The system according to claim 27, further comprising a memory that receives the subjective manifest refraction measurement of the eye.

29. The system according to claim 28, wherein the wavefront-based instrument comprises the memory.

30. The system according to claim 28, wherein the treatment planner comprises the memory.

31. The system according to claim 28, wherein the surgical device comprises the memory.

32. The system according to claim 28, further comprising:
a processor that calculates a difference between the wavefront-derived objective optical manifest refraction measurement and the subjective manifest refraction measurement; and
a prompting mechanism that presents a signal if the difference exceeds a threshold.

33. The system according to claim 32, further comprising an input that receives a physician acknowledgement of the difference.

34. The system according to claim 32, further comprising an input that receives a re-measurement instruction from the physician.
